(19) United States
(12) Patent Application Publication
(10) Pub. No.: US 2011/0143622 A1
(43) Pub. Date: Jun. 16, 2011

(54) FLUORINATED ACRYLATE COMPOSITIONS

(75) Inventor: ANILKUMAR RAGHAVANPILLAI, Wilmington, DE (US)

(73) Assignee: E.I. DUPONT DE NEMOURS AND COMPANY, Wilmington, DE (US)

(21) Appl. No.: 12/965,944

(22) Filed: Dec. 13, 2010

Related U.S. Application Data
(60) Provisional application No. 61/286,969, filed on Dec. 16, 2009.
(60) Provisional application No. 61/286,983, filed on Dec. 16, 2009.
(60) Provisional application No. 61/287,008, filed on Dec. 16, 2009.

(51) Int. Cl. C08F 126/00; C07C 271/10; C07C 69/52; B05D 1/02; B05D 1/18; B05D 1/28; B32B 27/08; B32B 27/10; B32B 5/18; D04H 13/00

(52) U.S. Cl. 442/327; 560/160; 560/222; 526/288; 427/427.4; 427/430.1; 427/429; 427/428.01; 428/221; 428/476.3; 428/522; 428/516; 428/514; 428/319.3

(57) ABSTRACT

Disclosed are fluorinated acrylate compounds. The compounds are useful as monomers in the preparation of polymeric surfactants and additives for coating compositions or as treatment agents to impart various surface properties to substrates

### CROSS REFERENCE TO RELATED APPLICATIONS

This application is related to and claims the benefit of priority of U.S. Provisional Patent Application No. 61/286,969, U.S. Provisional Patent Application No. 61/286,983, and U.S. Provisional Patent Application No. 61/287,008, all of which were filed Dec. 16, 2009, and all of which are currently pending.

### FIELD OF THE INVENTION

This invention relates to the field of fluorinated acrylate compounds. The compounds are useful as monomers in the preparation of polymeric surfactants and additives for coating compositions or as treatment agents to impart various surface properties to substrates

### BACKGROUND

Polyfluorinated compositions are used in the preparation of a wide variety of surface treatment materials. Polyfluorinated compositions are typically made of perfluorinated carbon chains connected directly or indirectly to nonfluorinated functional groups such as hydroxyl groups, carboxylic acid groups, and halide groups. Various compositions made from perfluorinated compounds or polymers are known to be useful as surfactants or treating agents to provide surface effects or to alter surface properties of substrates. Surface properties and effects include repellency to moisture, soil, and stains, and other effects, which are particularly useful for fibrous substrates and other substrates such as hard surfaces. Many such surfactants and treating agents are fluorinated polymers or copolymers.

Most commercially available fluorinated polymers useful as treating agents for altering surface properties of substrates contain predominantly eight or more carbons in the perfluoroalkyl chain to provide the desired properties. However, polymers containing shorter chain perfluoroalkyls have traditionally not been successful commercially for providing surface properties to treated substrates.

It is desirable to improve particular surface properties and to increase the fluorine efficiency; i.e., boost the efficiency or performance of treating agents so that lesser amounts of the expensive fluorinated composition are required to achieve the same level of performance, or so that better performance is achieved using the same level of fluorine. It is desirable to reduce the chain length of the perfluoroalkyl groups thereby reducing the amount of fluorine present, while still achieving the same or superior surface properties.

There is a need for compositions with better fluorine efficiency which significantly improve the repellency and stain resistance of fluorinated treating agents for substrates while using lower levels of fluorine.

### SUMMARY OF THE INVENTION

One aspect of the present invention is a compound of Formula (I):

R_{f}—S—(CH₂CH₂)-[G-C(O)]ₓ—NH—[(CH₂CH₂)—O]ₓ—C(O)—C(R)═CH₂ (I)

wherein R_{f} is perfluorinated alkyl, R is H, Cl, F or CH₃, x is 0 or 1, and G is O or NH.

Another aspect of the present invention is a polymer comprising one or more monomer units derived from a compound of Formula (I). The polymer can additionally comprise one or more monomer units derived from alkyl (meth)acrylate.

Another aspect of the present invention is a method of treating a substrate with a polymer comprising:

(a) about 20% to about 95% by weight of one or more monomer units derived from a compound of Formula (I):

R_{f}—S—(CH₂CH₂)-[G-C(O)]ₓ—NH—[(CH₂CH₂)—O]ₓ—C(O)—C(R)═CH₂ (I)

wherein
R_{f} is perfluorinated alkyl,

R is H, Cl, For CH₃,
x is 0 or 1, and
G is O or NH; and

(b) about 5% to about 80% of one or more monomer units derived from alkyl (meth)acrylate.

### DETAILED DESCRIPTION

Disclosed herein are fluorinated acrylate compounds, in particular perfluorobutyl and perfluorohexyl thioether derivatives, which can be used as intermediates or monomers useful for the manufacture of surface protection agents. Examples of such surface protection agents include, for example, surface active agents, compositions providing surface effects to various substrates, and compositions having numerous other uses for which a perfluorinated end group provides special surface-modifying properties. Examples of surface effects provided to substrates, in particular fibrous and hard surface substrates, treated with the surface protection agents include water repellency, oil repellency, soil repellency, and other surface effects.

Provided is a compound of Formula (I)

R_{f}—S—(CH₂CH₂)-[G-C(O)]ₓ—NH—[(CH₂CH₂)—O]ₓ—C(O)—C(R)═CH₂ (I)

wherein R_{f} is perfluorinated alkyl,

R is H, Cl, For CH₃,

x is 0 or 1, and

G is O or NH.

By “perfluorinated alkyl” is meant an alkyl group up to and including 12 carbons where all the hydrogen atoms have been replaced with fluorine atoms. Examples of such alkyl groups include methyl, ethyl, propyl, isopropyl, butyl, s-butyl, isobutyl, pentyl, neopentyl, hexyl, heptyl, isoheptyl, 2-ethylhexyl, cyclohexyl and octyl. The R_{f} group can be linear, branched, or cyclic, but is typically linear. In one embodiment, R_{f} is a 2 to 8 carbon, or 4 to 6 carbon perfluorinated alkyl. Typically R_{f} is n-C₄F₉ or n-C₆F₁₃.

The compounds can be synthesized by reacting compounds of the Formula R_{f}—I with either SH—(CH₂CH₂)—OH or SH—(CH₂CH₂)—NH₂ to form R_{f}—S—(CH₂CH₂)—OH or R_{f}—S—(CH₂CH₂)—NH₂

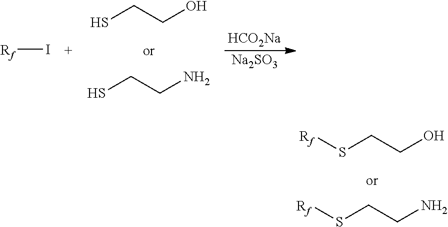

Compounds of Formula I where x is 1 can then be prepared by reacting the corresponding mercapto alcohol or amine with the appropriate isocyano-acrylate:

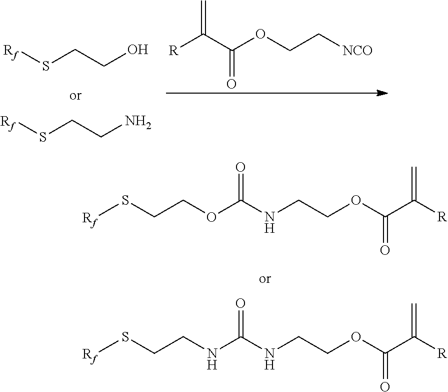

Compounds of Formula I where x is 0 can be prepared by reacting the mercapto amine with an acryloyl chloride:

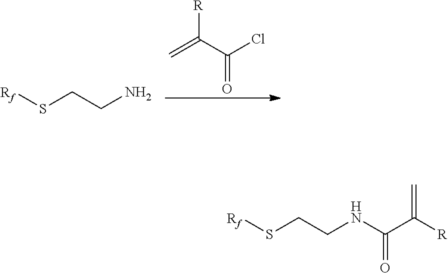

The reactions are typically performed in an organic solvent, such as methylene chloride or ethers. The compounds can be separated or purified by any method known in the art, such as extraction, which can be facilitated by the use of the appropriate organic solvent as reaction medium. Impurities can be removed by extraction with water to provide a separated organic layer, drying the separated organic layer over a desiccant, and then isolating the desired product by removing the organic solvent.

The compounds disclosed herein are useful as intermediates in the preparation of surface treatment chemicals and polymers. They are particularly useful as co-monomers with monomers such as (meth)acrylates to prepare polymeric surface treatment chemicals.

Provided is a polymer comprising one or more monomer units derived from a compound of Formula (I):

R_{f}—S—(CH₂CH₂)-[G-C(O)]ₓ—NH—[(CH₂CH₂)—O]ₓ—C(O)—C(R)═CH₂ (I)

wherein

R_{f} is perfluorinated alkyl,

R is H, Cl, For CH₃,

X is 0 or 1, and

G is O or NH.

The term “polymer” as used herein encompasses homopolymers, copolymers, terpolymers, or oligomers.

The term “alkyl (meth)acrylate” as used herein encompasses alkyl esters of methacrylic acid and acrylic acid unless specifically stated otherwise, and refers to the general class of ethylenically unsaturated monomers derived from methacrylic and acrylic acids and alkyl and substituted-alkyl esters thereof. For instance, hexyl (meth)acrylate encompasses both hexyl acrylate and hexyl methacrylate.

The polymer can further comprise additional monomer units, such as one or more monomer units derived from alkyl (meth)acrylate. The polymers comprising monomer units derived from compounds of Formula (I) and one or more monomer units derived from alkyl (meth)acrylate typically comprise about 5% to about 80% by weight, or about 40% to about 60%, or about 50% of one or more alkyl (meth)acrylate monomer, and 20% to about 95% by weight, or about 60% to about 40% of one or more monomer of Formula (I). Additional co-monomers can also be used. The alkyl (meth)acrylate monomers typically contain a linear, branched or cyclic alkyl group of from about 6 to about 18 carbons. Suitable alkyl (meth)acrylate monomers include stearyl (meth)acrylate, 2-ethylhexyl (meth)acrylate, hexyl (meth)acrylate, cyclohexyl (meth)acrylate, lauryl (meth)acrylate, tridecyl (meth)acrylate, or a mixture thereof, and typically are include one or more of stearyl (meth)acrylate and 2-ethylhexyl (meth)acrylate.

The polymers comprising monomer units derived from compounds of Formula (I) and optionally one or more monomer units derived from alkyl (meth)acrylate can optionally contain at least one additional monomer units derived from the following compounds copolymerized in the following percentages by weight:

(a) from about 1% to about 35% vinylidene chloride, vinyl chloride, or vinyl acetate, or a mixture thereof;

(b) from about 0.5% to about 25% of at least one compound selected from the group consisting of styrene, methyl-substituted styrene, chloromethyl-substituted styrene, 2-hydroxyethyl (meth)acrylate, ethylenediol di(meth)acrylate, N-methyloyl (meth)acrylamide, C1-C5 alkyl (meth)acrylate, 2-acrylamido-2-methyl-1-propane sulfonic acid (AMPS), and a compound of Formula (III):

R⁴(OCH₂CH₂)_{q}O—C(O)—C(R⁵)═CH₂ (III)

wherein

q is 2 to about 10;

R⁴ is H, a C₁ to C₄ alkyl, or CH₂═C(R)C(O)—O—; and

R⁵ is H, Cl, F or CH₃; or

(c) from about 0.5% to about 10% of at least one compound of Formula (IV)

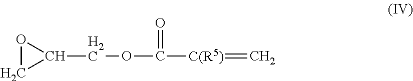

wherein

R⁵ is H, Cl, F or CH₃; or any combination thereof.

The polymers comprising monomer units derived from compounds of Formula (I) and optionally one or more monomer units derived from alkyl (meth)acrylate can additionally comprise from about 1% to about 35% by weight of monomer units derived from vinylidene chloride, vinyl chloride, vinyl acetate, or a mixture thereof. Alternately, the polymers can comprise from about 10% to about 30% of monomer units derived from vinylidene chloride, vinyl chloride, or a mixture thereof.

The polymers comprising monomer units derived from compounds of Formula (I) and optionally one or more monomer units derived from alkyl (meth)acrylate can alternatively comprise from about 0.5 weight % to about 25 weight %, of one or more monomer units derived from compounds selected from the group consisting of: styrene, methyl-substituted styrene, chloromethyl-substituted styrene, 2-hydroxyethyl (meth)acrylate, ethylenediol di(meth)acrylate, N-methyloyl (meth)acrylamide, C1-C5 alkyl (meth)acrylate, and compounds of Formula (III) as defined previously. They can also alternatively comprise from about 3% to about 10% on a weight basis, of monomer units derived from 2-hydroxyethyl (meth)acrylate, ethylenediol di(meth)acrylate, N-methyloyl (meth)acrylamide, and compounds of Formula (III) wherein q is 4 to 10 and R⁴ is hydrogen.

The polymers comprising monomer units derived from compounds of Formula (I) and optionally one or more monomer units derived from alkyl (meth)acrylate can also alternatively comprise monomer units derived from component (a) and component (b), each as defined above.

The polymers comprising monomer units derived from compounds of Formula (I) and optionally one or more monomer units derived from alkyl (meth)acrylate can also alternatively comprise optionally monomer units derived from component (a) as defined above; and further comprise monomer units derived from component (c) which is from about 0.5% to about 10% of one or more monomers units derived from Formula (IV) as defined above. Component (c) can comprise from about 0.5% to about 3% on a weight basis, of the polymer formulation.

Emulsion polymerization can be employed to prepare the polymers. The polymerization is carried out in a reactor fitted with a stirrer and external means for heating and cooling the charge. The monomers to be polymerized together are emulsified in an aqueous solution containing a suitable surfactant, and optionally an organic solvent, to provide an emulsion concentration of monomers 5% to 50% by weight. The temperature is raised to about 40° C. to about 70° C. to effect polymerization in the presence of an added free radical initiator. A suitable initiator is any of the commonly known agents for initiating the polymerization of an ethylenically unsaturated compound. Commonly known initiators include 2,2′-azodi-isobutyramidine dihydrochloride; 2,2′-azodiisobutyro-nitrile; 2,2′-azobis(2-methylpropionamidine) dihydrochloride and 2,2′ azobis(2,4-dimethyl-4-methoxyvaleronitrile. The concentration of added initiator is usually 0.1 to about 2 weight percent, based on the weight of the monomers to be polymerized. To control molecular weight of the resulting polymer, a chain-transfer agent, such as an alkylthiol of 4 to about 18 carbon atoms, is optionally present during polymerization.

The surfactants used in the polymerization are any of those cationic, anionic, nonionic and amphoteric surfactants commonly used for preparing aqueous emulsions. Suitable cationic agents include, for example, dodecyltrimethylammonium acetate, trimethyltetradecylammonium chloride, hexadecyltrimethylammonium bromide, trimethyloctadecylammonium chloride, ethoxylated alkyl amine salts, and others. A suitable example of a suitable cationic surfactant is the chloride salt of an ethoxylated alkyl ammonium salt such as an 18-carbon alkylamine with 15 moles of ethylene oxide such as Ethoquad® 18/25 available from Akzo Nobel, Chicago, Ill. Nonionic surfactants which are suitable for use herein include condensation products of ethylene oxide with 12-18 carbon atom fatty alcohols, 12-18 carbon fatty acids, alkyl phenols having 8-18 carbon atoms in the alkyl group, 12-18 carbon atom alkyl thiols and 12-18 carbon atom alkyl amines. A suitable nonionic surfactant, if used in combination with the cationic surfactant, is an ethoxylated tridecyl alcohol surfactant such as Merpol® SE available from Stepan Company, Northfield, Ill. Suitable anionic surfactants which are used herein include alkyl carboxylic acids and their salts, alkyl hydrogen sulfates and their salts, alkyl sulfonic acids and their salts, alkyl ethoxy sulfates and their salts, alpha olefin sulfonates, alkylamidoalkylene sulfonates, and the like. Generally suitable are those wherein the alkyl groups have 8-18 carbon atoms. Especially suitable is an alkyl sulfate sodium salt where the alkyl group averages about 12 carbons, such as Supralate® Wage surfactant, available from Witco Corporation, Greenwich, Conn.

Alternatively, solution polymerization in a suitable organic solvent can be used to prepare the polymer compositions. Solvents which can be used for the polymerization include, but are not limited to: ketones, for example, acetone, methyl ethyl ketone (MEK), and methyl isobutyl ketone (MIBK); alcohols, for example isopropanol; esters, for example butyl acetate; and ethers, for example, methyl t-butyl ether. The monomers to be polymerized together are charged to a reactor as disclosed above, together with a solvent. Typically the total monomer concentration in the organic solvent or mixture of organic solvents can be from about 20% to about 70% by weight of the solution. The temperature is raised to about 60° C. to about 90° C. to effect polymerization in the presence of at least one initiator, used in a quantity of 0.1 to 2.0 weight % based on the total weight of monomers. Initiators useful to effect polymerization in solution include: peroxides, for example benzoyl peroxide and lauryl peroxide; and azoic compounds for example, 2,2′-azobisisobutyronitrile, and 2,2′-azobis(2-methylbutyronitrile). To control molecular weight, optionally a chain-transfer agent, such as an alkylthiol, disclosed above, can be used.

The polymers disclosed herein can be used in a method of treating a substrate to impart oil repellency, water repellency, soil resistance, stain resistance, stain release, and wicking comprising contacting the substrate with the polymer. For fibrous substrates the method also imparts stain release and wicking properties to the substrate. The polymers are applied directly to a substrate. The polymers are applied alone or in admixture with dilute nonfluorinated polymers, or with other treatment agents or finishes. The polymers can be applied at a manufacturing facility, retailer location, or prior to installation and use, or at a consumer location.

Provided is a method of treating a substrate with a polymer comprising:
(a) about 20% to about 95% by weight of one or more monomer units derived from a compound of Formula (I):

R_{f}—S—(CH₂CH₂)-[G-C(O)]ₓ—NH—[(CH₂CH₂)—O]ₓ—C(O)—C(R)═CH₂ (I)

wherein

R_{f} is perfluorinated alkyl,

R is H, Cl, For CH₃,

x is 0 or 1, and

G is O or NH; and
(b) about 5% to about 80% of one or more monomer units derived from alkyl (meth)acrylate.

The polymers can be used as an additive during the manufacture of substrates. They can be added at any suitable point during manufacture. For example, in the case of paper, the polymers are added to the paper pulp in a size press. Preferably, from about 0.3% to about 0.5% by weight of polymers are added to paper pulp, based on the dry solids of the polymer and dry paper fiber.

Alternatively, the polymers can be applied to hard surface substrates by contacting the substrate with the polymers by conventional methods including, but not limited to, by brush, spray, roller, doctor blade, wipe, immersion, dip techniques, foam, liquid injection, and casting. Optionally, more than one coat can be applied, particularly on porous surfaces. When used on stone, tile and other hard surfaces, the polymers are typically diluted with water to give an application solution, optionally containing other additives, having from about 0.1% by weight to about 20% by weight, preferably from about 1.0% by weight to about 10% by weight, and most preferably from about 2.0% by weight to about 5.0% by weight, of the application solution based on solids. (i.e., all components except water or solvent). The coverage as applied to a substrate is about 100 g of application solution per sq meter (g/m²) for semi-porous substrates (e.g. limestone) and about 200 g/m² for porous substrates (e.g. Saltillo). Preferably the application results in from about 0.1 g/m² to about 2.0 g/m² of solids being applied to the surface.

The polymers are generally applied to fibrous substrates, such as nonwovens, fabrics, and fabric blends, as aqueous emulsions, dispersions, or solutions by spraying, dipping, padding, or other well-known methods. The polymers are generally diluted with water to concentrations of about 5 g/L to about 100 g/L, preferably about 10 g/L to about 50 g/L, based upon the weight of the fully formulated emulsion. After excess liquid has been removed, for example by squeeze rolls, the treated fabric is dried and then cured by heating, for example, to 110° C. to 190° C., for at least 30 seconds, typically from about 60 to about 180 seconds. Such curing enhances repellency and durability. While these curing conditions are typical, some commercial apparatus may operate outside these ranges because of its specific design features.

The polymers can contact the substrate as such, or in combination with other finishes or surface treating agents. The polymers optionally can be in combination with additional components such as treating agents or finishes to achieve additional surface effects, or additives commonly used with such agents or finishes. Such additional components comprise compounds or compositions that provide surface effects such as no iron, easy to iron, shrinkage control, wrinkle free, permanent press, moisture control, softness, strength, anti-slip, anti-static, anti-snag, anti-pill, stain repellency, stain release, soil repellency, soil release, water repellency, oil repellency, odor control, antimicrobial, sun protection, and similar effects, which are terms known to those skilled in the art. One or more such treating agents or finishes can be combined with the blended composition and applied to the fibrous substrate.

In particular for fibrous substrates, when textiles such as synthetic or cotton fabrics are treated, a wetting agent can be used, such as Alkanol® 6112 wetting agent available from E.I. du Pont de Nemours and Company, Wilmington, Del. When cotton or cotton-blended fabrics are treated, a wrinkle-resistant resin can be used such as Permafresh® EFC available from Omnova Solutions, Chester, S.C.

Other additives commonly used with such treating agents or finishes can also be present such as surfactants, pH adjusters, cross linkers, wetting agents, wax extenders, and other additives known by those skilled in the art. Suitable surfactants include anionic, cationic, and nonionic. Preferred is an anionic surfactant such as sodium lauryl sulfonate, available as Duponol® WAQE from Witco Corporation, Greenwich, Conn. Examples of such finishes or agents include processing aids, foaming agents, lubricants, anti-stains, and the like. The polymer and optional additives and other components can be applied at a manufacturing facility, retailer location, or prior to installation and use, or at a consumer location.

Optionally a blocked isocyanate can be added to the polymers to further promote durability (i.e., as a blended isocyanate). An example of a suitable blocked isocyanate is Hydrophobol® XAN available from Ciba Specialty Chemicals, High Point, N.J. Other commercially available blocked isocyanates are also suitable for use. The desirability of adding a blocked isocyanate depends on the particular application for the treating agent. Typically, a blocked isocyanate is not necessary for satisfactory cross-linking between chains or bonding to the substrate. when added as a blended isocyanate, amounts up to about 20% by weight [based on the weight of the blended isocyanate are added.

Optionally, nonfluorinated extender compositions can be included in the application composition to obtain some combination of benefits. Examples of such an optional additional extender polymer composition include those disclosed in U.S. Pat. No. 7,344,758.

Substrates useful in the method disclosed above include hard surface substrates, nonwoven materials, and fibrous substrates. Suitable substrates, after being contacted with the polymers, typically have fluorine contents of from about 0.05% by weight to about 0.5% by weight.

Hard surface substrates include porous and non-porous mineral surfaces, such as glass, stone, masonry, concrete, unglazed tile, brick, porous clay and various other substrates with surface porosity. Specific examples of such substrates include unglazed concrete, brick, tile, stone including granite, limestone and marble, grout, mortar, statuary, monuments, composite materials such as terrazzo, and wall and ceiling panels including those fabricated with gypsum board.

Fibrous substrates include textiles, nonwovens, fabrics, fabric blends, carpet, wood, paper and leather. Textiles and fabrics comprise polyamides including but not limited to polyamide-6,6 (PA-66), polyamide-6 (PA-6), and polyamide-6,10 (PA-610), polyesters including but not limited to polyethylene terephthalate (PET), polytrimethylene terephthalate, and polybutylene terephthalate (PBT); rayon; cotton; wool; silk; hemp; and combinations thereof. Nonwoven materials include fibers of glass, paper, cellulose acetate and nitrate, polyamides, polyesters, polyolefins including bonded polyethylene (PE) and polypropylene (PP), and combinations thereof. Specific nonwovens include, for instance, polyolefins including PE and PP such as Tyvek® (flash spun PE fiber), Sontara® (nonwoven polyester), and Xavan® (nonwoven PP), Suprel®, a nonwoven spunbond-meltblown-spunbond (SMS) composite sheet comprising multiple layers of sheath-core bicomponent melt spun fibers and side-by-side bicomponent meltblown fibers, such as disclosed in U.S. Pat. No. 6,548,431, U.S. Pat. No. 6,797,655 and U.S. Pat. No. 6,831,025, all such nonwovens being trademarked products of E.I. du Pont de Nemours and Company, Wilmington, Del.; nonwoven composite sheets comprising sheath-core bicomponent melt spun fibers, such as disclosed in U.S. Pat. No. 5,885,909; other multi-layer SMS nonwovens that are known in the art, such as PP spunbond-PP meltblown-PP spunbond laminates; nonwoven glass fiber media that are known in the art and as disclosed in U.S. Pat. No. 3,338,825, U.S. Pat. No. 3,253,978, and references cited therein; and Kolon® (spunbond polyester, a trademarked product of Korea Vilene, Seoul, South Korea). The nonwoven materials include those formed by web forming processing including dry laid (carded or air laid), wet laid, spunbonded and melt blown. The nonwoven web can be bonded with a resin, thermally bonded, solvent bonded, needle punched, spun-laced, or stitch-bonded. The bicomponent melt spun fibers, referred to above, can have a sheath of PE and a core of polyester. If a composite sheet comprising multiple layers is used, the bicomponent melt-blown fibers can have a polyethylene component and a polyester component and be arranged side-by-side along the length thereof. Typically, the side-by-side and the sheath/core bicomponent fibers are separate layers in the multiple layer arrangement.

Suitable fibrous substrates for the methods disclosed herein include one or more materials selected from the group consisting of cotton, rayon, silk, wool, hemp, polyester, spandex, polypropylene, polyolefin, polyamide, aramid, and blends or combinations thereof. Suitable nonwovens include paper, cellulose acetate and nitrate, polyamides, polyesters, polyolefins, and combinations thereof. Most suitable nonwoven are bonded polyethylene, polypropylene, polyester, and combinations thereof.

The polymers disclosed above are useful to provide one or more of excellent water repellency, oil repellency, soil resistance, stain release, and wicking to treated substrates. They allow for the use of shorter perfluoroalkyl groups containing 6 or fewer fluorinated carbon atoms for increased fluorine efficiency in the protection of treated surfaces. The polymers also allows for the use of polymers having minimal environmental impact.

### EXAMPLES

### Materials and Methods

All solvents and reagents, unless otherwise indicated, were purchased from commercial sources (Alfa Aesar, WardHill, Mass., TCl America Organic Chemicals, Portland, or Sigma Aldrich, Milwaukee, Wis.), and used directly as supplied. The textile fabrics (cotton, polyester, nylon) were purchased from Textile Innovators Corporation, 100 Forest Street, Windsor, N.C. 27983. ¹H and ¹⁹F NMR spectra were recorded on a Brucker DRX400 or 500 Spectrometer. Chemical shifts have been reported in ppm relative to an internal reference (CDCl_{3′}, CFCl₃ or TMS).

Application onto Textile Fabrics:

Textile fabric was treated with the fluorinated polymer solutions using the following process. Polymer solutions were prepared in tetrahydrofuran to contain 2000 mg/kg of fluorine. The solutions were applied to fabric substrates by pipetting the polymer solution onto substrates to saturation. After application, the substrate was dried in air and cured at approximately 150° C. for about 2 minutes. The substrate was allowed to cool to room temperature before the oil and water repellency measurements were conducted.

Test Method 1—Water/Oil Repellency Test on treated Fabric

The water repellency of a substrate (textile fabric) was measured according to AATCC (American Association of Textile Chemists and Colorists) standard Test Method No. 193-2004 and the DuPont Technical Laboratory Method as outlined in the Teflon® Global Specifications and Quality Control Tests information packet. The test determines the resistance of a substrate to wetting by aqueous liquids. Drops of test liquids comprising water-alcohol mixtures of varying surface tensions were placed on the substrate and the extent of surface wetting was determined visually. Three drops of Test Liquid 1 are placed on the substrate. After 10 seconds, the drops are removed by using vacuum aspiration. If no liquid penetration or partial absorption (appearance of a darker wet patch on the substrate) is observed, the test is repeated with Test Liquid 2. The test is repeated with Test Liquid 3 and progressively higher Test Liquid numbers until liquid penetration (appearance of a darker wet patch on the substrate) is observed. The test result is the highest Test Liquid number that does not penetrate into the substrate. Higher scores indicate greater water repellency.

The composition of water repellency test liquids is shown in the Table 1 below.

TABLE 1

| | Composition, | Composition, |
|---|---|---|
| Water Repellency | Volume % | Volume % |
| Rating Number | Isopropyl Alcohol | Distilled Water |
| 1 | 2 | 98 |
| 2 | 5 | 95 |
| 3 | 10 | 90 |
| 4 | 20 | 80 |
| 5 | 30 | 70 |
| 6 | 40 | 60 |
| 7 | 50 | 50 |
| 8 | 60 | 40 |
| 9 | 70 | 30 |
| 10 | 80 | 20 |
| 11 | 90 | 10 |
| 12 | 100 | 0 |

### Oil Repellency Test

The oil repellency of a substrate (textile fabric, leather, carpet, etc.) was tested using a modification of AATCC standard Test Method No. 118, conducted as follows. A substrate is maintained for a minimum of 2 hours at 23° C.+20% relative humidity and 65° C.+10% relative humidity. A series of organic liquids, identified below in the Table 2, are then applied drop wise to the substrate. Beginning with the lowest numbered test liquid (Repellency Rating No. 1), one drop (approximately 5 mm in diameter or 0.05 mL volume) is placed on each of three locations at least 5 mm apart. The drops are observed for 30 seconds. If, at the end of this period, two of the three drops are still spherical in shape with no wicking around the drops, three drops of the next highest numbered liquid are placed on adjacent sites and similarly observed for 30 seconds. The procedure is continued until one of the test liquids results in two of the three drops failing to remain spherical to hemispherical, or wetting or wicking occurs. The oil repellency rating is the highest numbered test liquid for which two of the three drops remained spherical to hemispherical, with no wicking for 30 seconds. Higher scores indicate greater oil repellency.

TABLE 2

| Oil Repellency | |
|---|---|
| Rating Number | Test Solution |
| 0 | Fails NUJOL ® Purified Mineral Oil |
| 1 | NUJOL ®Purified Mineral Oil |
| 2 | 65/35 Nujol ®/n-hexadecane by volume at 21° C. |
| 3 | n-hexadecane |
| 4 | n-tetradecane |
| 5 | n-dodecane |
| 6 | n-decane |
| 7 | n-octane |
| 8 | n-heptane |

Nujol ® is a trademark of Plough, Inc., for a mineral oil having a Saybolt viscosity of 360/390 at 38° C. and a specific gravity of 0.880/0.900 at 15° C.

### Test Method 2—Contact Angle Measurements

A 1% by weight solution of the polymer in tetrahydrofuran was dip coated onto Mylar® polyethylene terephthalate films (Du Pont Teijin Films, Hopewell, Va. 23860). The films were then air or vacuum dried for 24 hours before measuring the contact angles. Contact angle (CA) measurements to determine the dynamic contact angles of both water and hexadecane on the surface were performed using a Goniometer. Ramë-Hart Standard Automated Goniometer Model 200 employing DROP image standard software and equipped with an automated dispensing system with 250 μl syringe was used, having an illuminated specimen stage assembly. The Goniometer camera was connected through an interface to a computer and this allowed the droplet to be visualized on a computer screen. The horizontal axis line and the cross line could both be independently adjusted on the computer screen using the software.

To determine the contact angle of the test fluid on the sample, approximately one drop of test fluid was dispensed onto the sample using a 30 μL pipette tip of an automated dispensing system to displace a calibrated amount of the test fluid. For water measurements deionized water was employed, and for oil measurements, hexadecane was suitably employed. Prior to contact angle measurement, the sample was placed on the sample stage. The vertical vernier was adjusted to align the horizontal line (axis) of the eye piece coincident to the horizontal plane of the sample. The horizontal position of the stage relative to the eye piece was placed to view one side of the test fluid droplet interface region at the sample interface.

Horizontal and cross lines were adjusted via the software after leveling the sample via stage adjustment, and the computer calculated the contact angle based upon modeling the drop appearance. The initial contact angle is that angle determined immediately after dispensing the test fluid to the sample surface. Initial contact angles above 30 degrees are indicators of effective water and oil repellency. Contact angle can be measured after the droplet has been added to a surface (advancing contact angle, abbreviated “Adv CA”) or after the droplet has been partially withdrawn from a surface (receding contact angle, abbreviated “Rec CA”).

### Preparation of C₄F₉SCH₂CH₂OH

This compound was prepared using the method disclosed in in Magnier, E.; Tordeux, M.; Goumont, R.; Magder, K.; Wakselman, C. J. Fluorine Chem. 124 (2003), pp. 55-59.

A mixture of DMF (20 mL) and water (4 mL) was degassed, and added to it was 2-mercaptoethanol (1.54 g, 20.0 mmol) followed by perfluorobutyl iodide (6.7 g, 20.0 mmol). Next, HCO₂Na (1.47 g, 22.0 mmol) and Na₂SO₃(2.7 g, 22.0 mmol) was rapidly added to the solution with stirring. The mixture was stirred at room temperature overnight. Water (100 mL) and ether (100 mL) were added. The organic layer was separated and the aqueous layer extracted with ether (3×50 mL). The combined ether layer was washed with 2% HCl (1×100 mL), water (1×100 mL) and dried over anhydrous MgSO₄. The solvent was removed under vacuum followed by drying, resulting in a brownish oil. The product was analyzed by NMR and determined to be C₄F₉SCH₂CH₂OH (2.7 g, 9.1 mmol, 46%): ¹H NMR (CDCl₃): δ 3.81 (t, J=6.0 Hz, 2H), 3.06 (t, J=6.0 Hz, 2H), 1.8 (bs, 1H). ¹⁹F NMR (CDCl3): δ −81.4 (m, 3F), −87.3 (m, 2F), −121.0 (m, 2F), −125.8 (m, 2F).

### Preparation of C₄F₉SCH₂CH₂NH₂

A mixture of DMF (20 mL) and water (4 mL) was degassed, and added to it was 2-aminoethanethiol (1.5 g, 20.0 mmol) followed by perfluorobutyl iodide (6.7 g, 20.0 mmol). Next, HCO₂Na (1.47 g, 22.0 mmol) and Na₂SO₃(2.7 g, 22.0 mmol) was rapidly added to the solution with stirring. The mixture was stirred at room temperature overnight. Water (100 mL) and ether (100 mL) were added. The organic layer was separated and the aqueous layer extracted with ether (3×50 mL). The combined ether layer was washed with 2% HCl (1×100 mL), water (1×100 mL) and dried over anhydrous MgSO₄. The solvent was removed under vacuum followed by drying, resulting in a yellow oil. The product was analyzed by NMR and determined to be C₄F₉SCH₂CH₂NH₂ (4.6 g, 15.6 mmol, 78%). ¹H NMR (CDCl₃): δ 2.98 (bt, 4H), 1.3 (bs, 2H). ¹⁹F NMR (CDCl3): δ −81.4 (m, 3F), −87.3 (m, 2F), −121.0 (m, 2F), −125.8 (m, 2F).

### Preparation of C₆F₁₃SCH₂CH₂NH₂

A similar procedure as disclosed for compound 2 was followed using perfluorohexyl iodide (10.0 g, 22.4 mmol) and 2-aminoethanethiol (1.7 g, 22.4 mmol), which provided compound 2 as a pale yellow oil (5.8 g, 14.6 mmol, 65%). ¹H NMR (CDCl₃): δ 2.97 (bt, 4H), 1.28 (bs, 2H). ¹⁹F NMR (CDCl3): δ −81.3 (m, 3F), −87.2 (m, 2F), −120.2 (m, 2F), −121.7 (m, 2F)-123.2 (m, 2F), −126.4 (m, 2F).

### Preparation of C₄F₉SCH₂CH₂NHC(O)C(Me)═CH₂

A solution of methacryloyl chloride (0.353 g, 3.38 mmol) in methylene chloride (10 mL) was added dropwise to a mixture of C₄F₉SCH₂CH₂NH₂ (1.0 g, 3.38 mmol) and triethylamine (0.341 g, 3.38 mmol) in methylene chloride (10 mL) that was kept at 0° C. The reaction mixture was stirred 8 hours at ambient temperature. Water (20 mL) was added to the reaction mixture and the organic layer was separated and washed with 1N HCl (2×20 mL), sat. NaHCO₃ (2×20 mL) and brine (1×20 mL). The organic layer separated and dried over anhydrous MgSO₄. Removal of the solvent under reduced pressure followed by drying produced a pale yellow oil (1.0 g, 2.75 mmol, 82%): ¹H NMR (CDCl₃): δ 6.27 (bs, 1H), 5.64 (t, J=1 Hz, 1H), 5.30 (m, 1H), 3.53 (q, J=6.4 Hz, 2H), 3.08 (t, J=6.4 Hz, 2H), 1.89 (t, J=1.0 Hz, 3H). ¹⁹F NMR (CDCl3): δ −81.5 (m, 3F), −87.3 (m, 2F), −121.1 (m, 2F), −125.9 (m, 2F).

### Preparation of C₆F₁₃SCH₂CH₂NHC(O)C(Me)═CH₂

A similar procedure as disclosed for the synthesis of C₄F₉SCH₂CH₂NHC(O)C(Me)═CH₂ was followed. C₆F₁₃SCH₂CH₂NH₂ (1.0 g, 2.53 mmol) was reacted with methacryloyl chloride (0.291 g, 2.53 mmol) followed by aqueous work-up provided a pale yellow oil (1.0 g, 2.15 mmol, 85%): ¹H NMR (CDCl₃): δ 6.11 (bs, 1H), 5.64 (t, J=1 Hz, 1H), 5.30 (m, 1H), 3.54 (q, J=6.4 Hz, 2H), 3.07 (t, J=6.4 Hz, 2H), 1.90 (t, J=1.0 Hz, 3H). ¹⁹F NMR (CDCl3): δ −81.2 (m, 3F), −86.8 (m, 2F), −120.1 (m, 2F), −121.7 (m, 2F), −123.1 (m, 2F), −126.5 (m, 2F).

### Example 1

### C₄F₉SCH₂CH₂OC(O)NHCH₂CH₂OC(O)C(Me)═CH₂

A 3-necked 100 mL flask fitted with a nitrogen tee, septa and stopper was charged with anhydrous diethyl ether (5 mL), and C₄F₉SCH₂CH₂OH (0.3 g, 0.92 mmol). 2-isocyanatoethyl methacrylate (0.143 g, 0.92 mmol) was added to the solution was added drop wise. After the addition was complete, a catalytic amount of dibutyltin dilaurate (0.003 g, 0.004 mmol) was added and the mixture stirred for 12 hours at room temperature. Removal of the solvent followed by repeated washing with hexane-diethyl ether (3:1) and drying produced the desired compound as a pale yellow oil (0.4 g, 0.89 mmol). ¹H NMR (CDCl₃): δ 6.14 (bs, 1H), 5.62 (m, 1H), 4.35 (t, J=6.0 Hz, 2H), 4.26 (t, J=6.4 Hz, 2H), 3.51 (t, J=6.0 Hz, 2H), 2.95 (t, J=6.0 Hz, 2H), 1.97 (s, 3H). ¹⁹F NMR (CDCl3): δ −81.4 (m, 3F), −87.6 (m, 2F), −121.0 (m, 2F), −125.8 (m, 2F).

### Example 2

### C₄F₉SCH₂CH₂NHC(O)NHCH₂CH₂OC(O)C(Me)═CH₂

The procedure in Example 1 was followed using C₄F₉SCH₂CH₂NH₂ (0.4, 1.35 mmol) with 2-isocyanatoethyl methacrylate (0.19 g, 1.23 mmol) in diethylether (10.0 mL) to produce a pale yellow oil (0.55 g, 1.22 mmol): ¹H NMR (CDCl₃): δ 6.14 (bs, 1H), 5.62 (m, 1H), 5.05 (bs, 1H), 4.91 (bs, 1H), 4.25 (t, J=6.4 Hz, 2H), 3.64 (2 merging triplets, 4H), 3.13 (t, J=6.0 Hz, 2H), 1.96 (s, 3H). ¹⁹F NMR (CDCl3): δ −81.3 (m, 3F), −87.2 (m, 2F), −121.0 (m, 2F), −125.7 (m, 2F).

### Example 3

### C₆F₁₃SCH₂CH₂NHC(O)NHCH₂CH₂OC(O)C(Me)═CH₂

The procedure in Example 1 was followed using C₆F₁₃SCH₂CH₂NH (1 g, 2.53 mmol) with 2-isocyanatoethyl methacrylate (0.357 g, 2.30 mmol) in diethylether (10.0 mL) to produce a pale yellow oil (1.1 g, 1.22 mmol, 2.0 mmol, 87%): ¹H NMR (CDCl₃): δ 6.13 (t, J=1.0 Hz, 1H), 5.62 (t, J=1.0 Hz, 1H), 5.10 (bt, 1H), 4.97 (bt, 1H), 4.25 (dt, J=6.4, 1.0 Hz, 2H), 3.52 (2 merging triplets, 4H), 3.13 (t, J=6.0 Hz, 2H), 1.96 (s, 3H). ¹⁹F NMR (CDCl3): δ −81.2 (m, 3F), −86.9 (m, 2F), −120.1 (m, 2F), −121.7 (m, 2F), −123.1 (m, 2F), −126.5 (m, 2F).

### Example 4

### C₄F₉SCH₂CH₂OC(O)NHCH₂CH₂OC(O)C(Me)═CH₂/stearyl methacrylate polymer

In a 25 mL 3-necked flask fitted with a N₂ purge and condenser was added methylethyl ketone (MEK) (6 mL). The solvent was degassed 5 minutes. The compound prepared in Example 1, C₄F₉SCH₂CH₂OC(O)NHCH₂CH₂OC(O)C(Me)═CH₂, (0.4 g, 0.88 mmol) and stearyl methacrylate (0.299 g, 0.886 mmol) were then added to the flask. To the stirring solution was then added Vazo®67 (0.019 g) and the mixture was quickly placed into a preheated oil bath kept at 65° C. The progress of the polymerization was monitored by GPC. By 20 hours complete conversion of the monomers to the polymer was noticed. The reaction mixture was cooled to room temperature, methanol (10 mL) was added, and the solvent decanted from the precipitated polymer. The polymer was dried, re-dissolved in MEK (6 mL) and re-precipitated with methanol (10 mL). Upon removal of the solvent and drying a white solid (0.375 g was obtained. The polymer was applied to cotton and nylon fabrics and tested for water/oil repellency using Test Method 1. The test results are in Table 3. The polymer was also applied to Mylar® polyethylene terephthalate film and contact angles were measured according to Test Method 2. The test results are in Table 4.

### Example 5

### C₄F₉SCH₂CH₂NHC(O)C(Me)═CH₂/stearyl methacrylate polymer

The procedure of Example 4 was followed for the polymerization of C₄F₉SCH₂CH₂NHC(O)C(Me)═CH₂ (1.0 g, 2.75 mmol) with stearyl methacrylate (SMA) (0.93 g, 0.864 mmol) in MEK (6 g), to produce the desired polymer as a white solid (1.0 g). The polymer was applied to cotton and nylon fabrics and tested for water/oil repellency using Test Method 1. The test results are in Table 3. The polymer was also applied to Mylar® polyethylene terephthalate film and contact angles were measured according to Test Method 2. The test results are in Table 4.

### Example 6

### C₆F₁₃SCH₂CH₂NHC(O)C(Me)═CH₂/stearyl methacrylate polymer

The procedure of Example 4 was followed for the polymerization of C₆F₁₃SCH₂CH₂NHC(O)C(Me)═CH₂ (1.0 g, 2.16 mmol) with stearyl methacrylate (0.73 g, 2.16 mmol) in MEK (6 g) to produce the polymer as an off-white solid (0.980 g). The polymer was applied to cotton and nylon fabrics and tested for water/oil repellency using Test Method 1. The test results are in Table 3. The polymer was also applied to Mylar® polyethylene terephthalate film and contact angles were measured according to Test Method 2. The test results are in Table 4.

### Example 7

### C₄F₉SCH₂CH₂NHC(O)NHCH₂CH₂OC(O)C(Me)═CH₂/stearyl methacrylate polymer

The procedure of Example 4 was followed for the polymerization of C₄F₉SCH₂CH₂NHC(O)NHCH₂CH₂OC(O)C(Me)═CH₂ (0.55 g, 1.22 mmol) from Example 2 with stearyl methacrylate (0.412 g, 1.22 mmol) in MEK (6 g) to produce the polymer as a pale yellow solid (0.660 g). The polymer was applied to cotton and nylon fabrics and tested for water/oil repellency using Test Method 1. The test results are in Table 3. The polymer was also applied to Mylar® polyethylene terephthalate film and contact angles were measured according to Test Method 2. The test results are in Table 4.

### Example 8

### C₄F₉SCH₂CH₂NHC(O)NHCH₂CH₂OC(O)C(Me)═CH₂/stearyl methacrylate polymer

The procedure of Example 4 was followed for the polymerization of C₄F₉SCH₂CH₂NHC(O)NHCH₂CH₂OC(O)C(Me)═CH₂ (01.1 g, 2.0 mmol) from Example 3 with stearyl methacrylate (0.676 g, 2.0 mmol) in MEK (6 g) to produce the polymer as a pale yellow solid (0.780 g). The polymer was applied to cotton and nylon fabrics and tested for water/oil repellency using Test Method 1. The test results are in Table 3. The polymer was also applied to Mylar® polyethylene terephthalate film and contact angles were measured according to Test Method 2. The test results are in Table 4.

### Comparative Example 9

### 6,2-Methacrylate/stearyl methacrylate polymer

The procedure of Example 4 was followed for the polymerization of 6,2-methacrylate [C₆F₁₃CH₂CH₂OC(O)C(Me)═CH₂] (0.86 g, 2.0 mmol) with stearyl methacrylate (0.676 g, 2.0 mmol) in MEK (6 g) to produce the polymer as a pale yellow solid (0.780 g). The polymer was applied to cotton and nylon fabrics and tested for water/oil repellency using Test Method 1. The test results are in Table 3. The polymer was applied to Mylar® polyethylene terephthalate film and contact angles were measured according to Test Method 2. The test results are in Table 4.

### Comparative Example 10

### 4,2-Methacrylate/stearyl methacrylate polymer

The procedure of Example 4 was followed for the polymerization of 4,2-methacrylate [C₄F₉CH₂CH₂OC(O)C(Me)═CH₂] (0.664 g, 2.0 mmol) with stearyl methacrylate (0.676 g, 2.0 mmol) in MEK (6 g) to produce the polymer as a pale yellow solid (0.672 g). The polymer was applied to cotton and nylon fabrics and tested for water/oil repellency using Test Method 1. The test results are in Table 3. The polymer was applied to Mylar® polyethylene terephthalate film and contact angles were measured according to Test Method 2. The test results are in Table 4.

TABLE 3
Water and oil repellency rating of polymers treated on fabric

| | |
|---|---|
| Cotton | Nylon |

| 2000 ppm of F on Fabric | Water | Oil | Water | Oil |
|---|---|---|---|---|
| Example 4 | 4 | 1 | 6 | 2 |
| Example 5 | 3 | 1 | 4 | 1 |
| Example 6 | 6 | 1 | 6 | 2 |
| Example 7 | 4 | 1 | 5 | 1 |
| Example 8 | 7 | 1 | 7 | 3 |
| Comparative Example 9 | 6 | 1 | 7 | 2 |
| Comparative Example 10 | 3 | 1 | 4 | 1 |
| Untreated Control | 0 | 0 | 0 | 0 |

TABLE 4
Water and hexadecane contact angles of polymers
treated on Mylar ® film

| | Contact angle^{a} | | | |
|---|---|---|---|---|
| On Mylar ® film (dip | Water | | Hexadecane | |
| coated) 1 wt % F | Adv | Rec | Adv | Rec |
| Example 4 | 114 ± 4 | 53 ± 2 | 69 ± 3 | 45 ± 2 |
| Example 5 | 117 ± 1 | 81 ± 1 | 65 ± 1 | 24 ± 1 |
| Example 6 | 124 ± 3 | 61 ± 2 | 83 ± 1 | 39 ± 2 |
| Example 7 | 120 ± 1 | 69 ± 1 | 67 ± 1 | 46 ± 1 |
| Example 8 | 122 ± 1 | 75 ± 2 | 69 ± 2 | 31 ± 1 |
| Comparative Example 9 | 120 ± 1 | 80 ± 1 | 69 ± 2 | 30 ± 1 |
| Comparative Example 10 | 91 ± 3 | 47 ± 1 | 58 ± 2 | 21 ± 2 |
| Mylar ® film Control (untreated) | 85 | 32 | 25 | 10 |

^{a}An average of 3 runs at different parts of the sample

1. A compound of Formula (I)
R_{f}—S—(CH₂CH₂)-[G-C(O)]ₓ—NH—[(CH₂CH₂)—O]ₓ—C(O)—C(R)═CH₂ (I)
wherein
R_{f} is perfluorinated alkyl,
R is H, Cl, For CH₃,
X is 0 or 1, and
G is O or NH.

2. The compound of claim 1 wherein x is 1.

3. The compound of claim 1 wherein x is 0.

4. The compound of claim 1 wherein R_{f} is a 4 to 6 carbon perfluorinated alkyl.

5. The compound of claim 1 wherein x is 1, G is O and R_{f} is n-C₄F₉ or n-C₆F₁₃.

6. The compound of claim 1 wherein R is CH₃.

7. A polymer comprising one or more monomer units derived from the compound of claim 1.

8. The polymer of claim 7 further comprising one or more monomer units derived from alkyl (meth)acrylate.

9. The polymer of claim 8 wherein the alkyl (meth)acrylate contains an alkyl group of from about 6 to about 18 carbons.

10. The polymer of claim 8 wherein the alkyl (meth)acrylate is stearyl (meth)acrylate, 2-ethylhexyl (meth)acrylate, hexyl (meth)acrylate, cyclohexyl (meth)acrylate, lauryl (meth)acrylate, tridecyl (meth)acrylate, or a mixture thereof.

11. The polymer of claim 8 comprising from about 20% to about 95% by weight of one or more monomer units derived from a compound of Formula (I) and from about 5% to about 80% by weight of the one or more monomer units derived from alkyl (meth)acrylate.

12. A method of treating a substrate comprising contacting the substrate with a polymer comprising:
(a) about 20% to about 95% by weight of one or more monomer units derived from a compound of Formula (I):
R_{f}—S—(CH₂CH₂)-[G-C(O)]ₓ—NH—[CH₂CH₂)—O]ₓ—C(O)—C(R)═CH₂ (I)
wherein
R_{f} is perfluorinated alkyl,
R is H, Cl, For CH₃,
x is 0 or 1, and
G is O or NH; and
(b) about 5% to about 80% of one or more monomer units derived from alkyl (meth)acrylate.

13. The method of claim 12 wherein the substrate is a fibrous substrate, a nonwoven material, or a hard surface substrate.

14. The method of claim 12 wherein the substrate is
(a) a fibrous substrate selected from the group consisting of textiles, fabrics, fabric blends, polyamides, polyesters, polyolefins, spandex, rayon, cotton, wool, silk, hemp, carpet, wood, paper, leather, and combinations thereof;
(b) a nonwoven material selected from the group consisting of fibers of glass, paper, cellulose acetate, nitrate, polyamides, polyesters, polyolefins, polyethylene, polypropylene, and combinations thereof; or
(c) a hard surface substrate of porous or non-porous mineral selected from the group consisting of glass, stone, masonry, concrete, unglazed tile, brick, porous clay, unglazed concrete, granite, limestone, marble, grout, mortar, statuary, monuments, terrazzo, and gypsum board.

15. The method of claim 12 wherein the polymer is contacted with the substrate by exhaustion, spray, foam, flex-nip, nip, pad, kiss-roll, beck, skein, winch, liquid injection, overflow flood, dip, brush, roll, spray, roller, doctor blade, wipe, casting, or immersion.

16. The method of claim 12 wherein the polymer is contacted with the substrate in the presence of
a) an agent providing at least one surface effect selected from the group consisting of no iron, easy iron, shrinkage control, wrinkle free, permanent press, moisture control, softness, strength, anti-slip, anti-static, anti-snag, anti-pill, stain repellency, stain release, soil repellency, soil release, water repellency, oil repellency, odor control, antimicrobial, and sun protection;
b) a surfactant, pH adjuster, cross linker, wetting agent, blocked isocyanate, wax extender, or hydrocarbon extender; or
c) a mixture thereof.

17. A substrate treated by the method of claim 12.

18. The substrate of claim 17 which is
(a) a fibrous substrate selected from the group consisting of textiles, fabrics, fabric blends, polyamides, polyesters, polyolefins, spandex, rayon, cotton, wool, silk, hemp, carpet, wood, paper, leather, and combinations thereof;
(b) a nonwoven material selected from the group consisting of fibers of glass, paper, cellulose acetate, nitrate, polyamides, polyesters, polyolefins, polyethylene, polypropylene, and combinations thereof; or
(c) a hard surface substrate of porous or non-porous mineral selected from the group consisting of glass, stone, masonry, concrete, unglazed tile, brick, porous clay, unglazed concrete, granite, limestone, marble, grout, mortar, statuary, monuments, terrazzo, and gypsum board.
